(19) United States
(12) Patent Application Publication
(10) Pub. No.: US 2012/0003171 A1
(43) Pub. Date: Jan. 5, 2012

(54) VOLUMIZING SMUDGE RESISTANT MASCARA COMPOSITION

(75) Inventors: Hy S. Bui, Piscataway, NJ (US); Mohamed Kanji, Edison, NJ (US); Anita Chon Tong, Westfield, NJ (US); Bruno Bavouzet, Gentilly (FR)

(73) Assignee: L'OREAL S.A., Paris (FR)

(21) Appl. No.: 13/139,149

(22) PCT Filed: Dec. 11, 2009
(86) PCT No.: PCT/US09/67728

§ 371 Date: Sep. 20, 2011

Related U.S. Application Data
(60) Provisional application No. 61/121,584, filed on Dec. 11, 2008.
(60) Provisional application No. 61/121,581, filed on Dec. 11, 2008.
(60) Provisional application No. 61/121,587, filed on Dec. 11, 2008.
(60) Provisional application No. 61/121,592, filed on Dec. 11, 2008.

(51) Int. Cl. A61K 8/92; A61Q 1/10

(52) U.S. Cl. 424/70.7; 424/70.11; 424/70.16

(57) ABSTRACT

The present invention is directed to a volumizing eye make-up composition having a creamy texture and feel containing: (a) at least one alkyl ethoxylated polymer wax; (b) at least one polar modified polymer; (c) water; (d) optionally, at least one non-volatile solvent; (e) optionally, at least one volatile solvent; and (f) at least one colorant, and wherein the composition preferably has a solids content of less than 40% by weight, based on the weight of the composition.

### FIELD OF THE INVENTION

The present invention generally relates to a novel mascara composition and method of making-up eye lashes. More particularly, the present invention relates to a self-emulsifying, volumizing and smudge resistant mascara composition.

### DISCUSSION OF THE BACKGROUND

It is well known in the industry that the primary way of making a mascara composition capable of volumizing eyelashes is to introduce high amounts of solids, such as pigments, waxes and polymers, into the composition. The problem with such high solids content mascaras is that they have a tendency to aggregate and clump on the eye lashes. Flaking is oftentimes yet another drawback to such volumizing formulations.

Also, when formulating water-containing mascara compositions, surfactants/emulsifiers are conventionally used in order to form a stable emulsion. This, in turn, adds to the overall cost of the product.

Therefore, it is an object of the present invention to provide a volumizing mascara composition which does not require a large amount of solids or latex film formers.

### SUMMARY OF THE INVENTION

The present invention relates to a mascara composition for use on eye lashes comprising:
(a) at least one alkyl ethoxylated polymer wax;
(b) at least one oil-soluble polar modified polymer and/or at least one oil-soluble high carbon polar modified polymer;
(c) water;
(d) optionally, at least one non-volatile solvent capable of solubilizing the polar modified polymers;
(e) optionally, at least one volatile solvent ; and
(f) optionally, at least one colorant.

Preferably, the composition has a solids content of less than about 40% by weight, based on the weight of the composition.

The present invention also relates to a mascara composition for use on eye lashes made by combining ingredients comprising:
(a) at least one alkyl ethoxylated polymer wax;
(b) at least one oil-soluble polar modified polymer and/or at least one oil-soluble high carbon polar modified polymer;
(c) water;
(d) optionally, at least one non-volatile solvent capable of solubilizing the polar modified polymers;
(e) optionally, at least one volatile solvent ; and
(f) optionally, at least one colorant.

Preferably, the composition has a solids content of less than about 40% by weight, based on the weight of the composition.

The present invention also relates to a mascara composition for use on eye lashes made by combining ingredients comprising:
(a) a reaction product of (i) at least one alkyl ethoxylated polymer wax and (ii) at least one oil-soluble polar modified polymer and/or at least one oil-soluble high carbon polar modified polymer;
(c) water;
(d) optionally, at least one non-volatile solvent capable of solubilizing the polar modified polymers;
(e) optionally, at least one volatile solvent ; and
(f) optionally, at least one colorant.

Preferably, the composition has a solids content of less than about 40% by weight, based on the weight of the composition.

The present invention also relates to methods of making up eye lashes involving applying the above-disclosed composition onto the eye lashes.

The present invention also relates to methods of volumizing eyelashes (that is, increasing eyelash volume) comprising applying the above-disclosed composition onto eyelashes in an amount sufficient to volumize the eyelashes.

It has been surprisingly and unexpectedly discovered that the above-disclosed composition, when applied onto a keratinous substrate, such as eye lashes, can make eye lashes appear more voluminous, without having to employ a large amount of solids in the composition.

It has also been surprisingly discovered that the composition of the present invention forms a stable, long wear emulsion, having a unique texture and feel, without having to employ a surfactant/emulsifier to form the emulsion. In addition, the composition possesses long wear properties without having to employ conventional latex or other film forming polymers.

### BRIEF DESCRIPTION OF THE DRAWINGS

FIG. 1 depicts the Storage Modulus of the Mascaras from Examples 1-4.

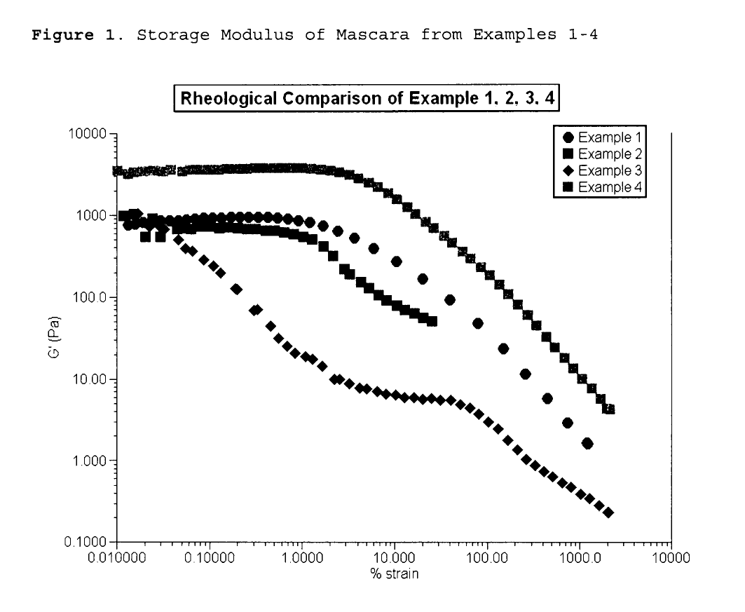

FIG. 2 depicts the Shear Viscosity of the Mascaras from Examples 1-4.

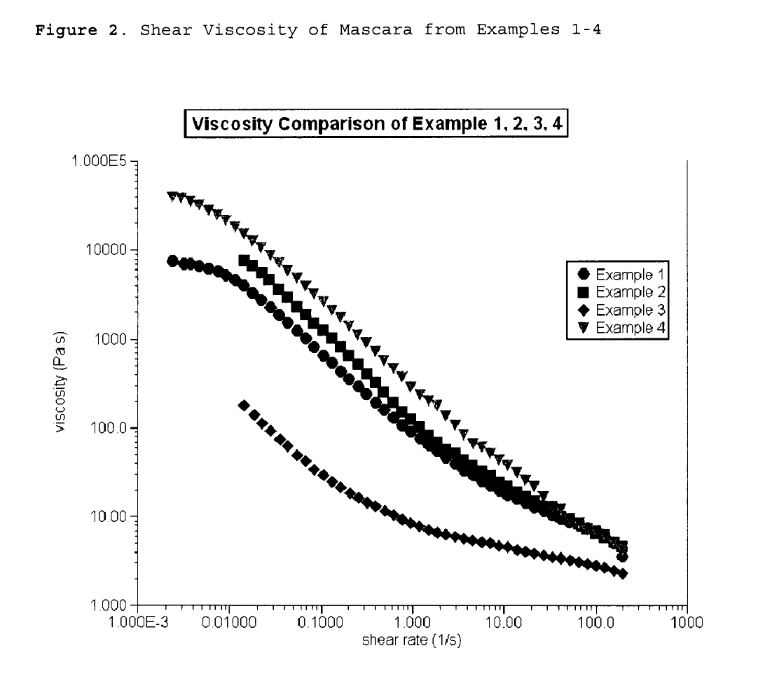

FIG. 3 depicts the Storage Modulus of the Mascaras from Example 7 and Comparative Examples 1 and 2.

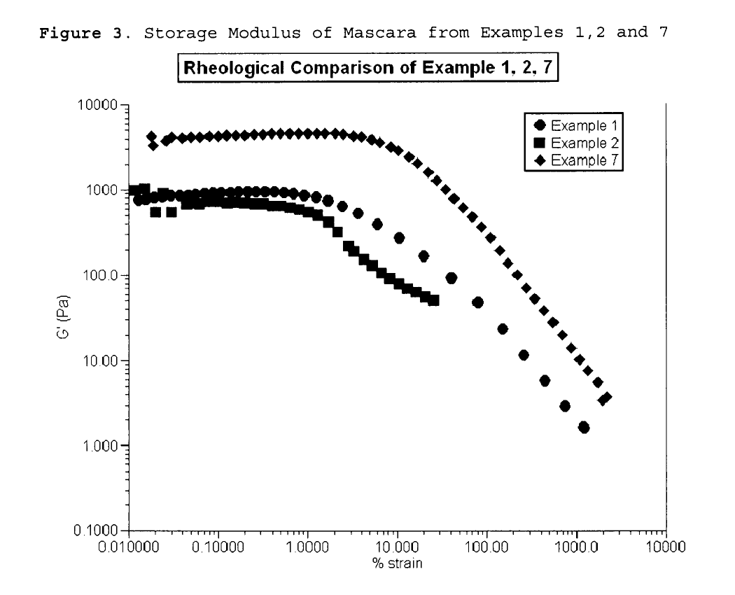

FIG. 4 depicts the Shear Viscosity of the Mascaras from Example 7 and Comparative Examples 1 and 2.

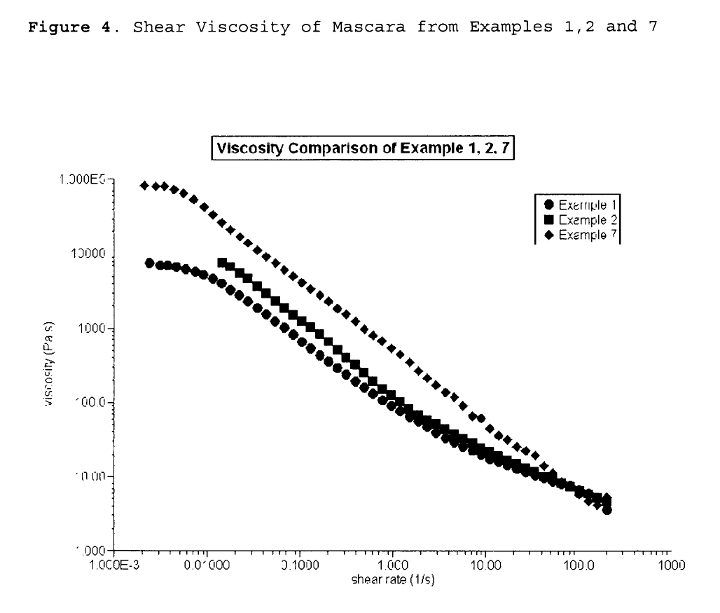

FIG. 5 depicts the Storage and Loss Modulii of the Mascara of Example 6 and of commercially available Maybelline Volume Express Washable Mascara.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

“Film former” or “film forming agent” or “film forming resin” as used herein means a polymer which, after dissolution in at least one solvent (such as, for example, water and organic solvents), leaves a film on the substrate to which it is applied, for example, once the at least one solvent evaporates, absorbs and/or dissipates on the substrate.

“Tackiness”, as used herein, refers to the adhesion between two substances. For example, the more tackiness there is between two substances, the more adhesion there is between the substances.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion, bending or leaning if the composition is in stick form, melting, or syneresis (or sweating). The stability is further tested by repeating the 8-week test at 37° C., 40° C., 45° C., 50° C., and under freeze-thaw conditions. A composition is considered to lack stability if in any of these tests an abnormality that impedes functioning of the composition is observed. The skilled artisan will readily recognize an abnormality that impedes functioning of a composition based on the intended application.

“Volatile”, as used herein, means having a flash point of less than about 100° C.

“Non-volatile”, as used herein, means having a flash point of greater than about 100° C.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Smudge proof” as used herein refers to the ability to repel sebum and permanence with respect to squalene. Smudge proof properties may be evaluated by any method known in the art for evaluating such properties. For example, a mascara composition may be applied to false eyelashes, which may then be placed in squalene for a certain amount of time, such as, for example, 20 minutes. Upon expiration of the pre-ascertained amount of time, the false eyelashes may be removed from the squalene and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in squalene for a certain amount of time. The amount of composition remaining on the skin after the pre-ascertained amount of time may then be evaluated and compared. For example, a composition may be smudge proof if a majority of the product is left on the wearer, e.g., eyelashes, skin, etc. In a preferred embodiment of the present invention, little or no composition is transferred from the wearer.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

Alkyl Ethoxylated Polymers

The compositions of the present invention comprise at least one alkyl ethoxylated polymer. The alkoxylated fatty alcohol can be present in the composition of the present invention in the water and/or oil phase.

Preferably, the alkyl ethoxylated polymer is chosen from di-alkyl, tri-alkyl- and combinations of di-alkyl and tri-alkyl substituted ethoxylated polymers. They can also be chosen from mono-alkyl, di-alkyl, tri-alkyl, tetra-alkyl substituted alkyl ethoxylated polymers and all combinations thereof. The alkyl group can be saturated or unsaturated, branched or linear and contain a number of carbon atoms preferably from about 12 carbon atoms to about 50 carbon atoms, including all ranges and subranges therebetween.

The alkyl substitution of the alkyl ethoxylated polymer includes mono-alkyl, di-alkyl, tri-alkyl and tetra-alkyl substitution of the polymer and combinations thereof. Suitable examples of mono alkyl substituted polymers include: Steareth-100 available as Brij 700 from Uniqema Inc., Pareth alcohols available as Performathox 450, 480 and 490 available from New Phase Technologies, Inc. Suitable examples of di-alkyl substituted polymers include PEG 120 methyl glucose dioleate available as Glutamate DOE-120 and Glucamate DOE-120 both from Chemron Corporation. Suitable examples of tri-alkyl substituted polymers include PEG 120 methyl glucose trioleate available as Glucamate LT from Chemron Corporation. Suitable examples of tetra-alkyl substituted polymers include PEG 150 pentaerythrityl tetrastearate available as Crothix from Croda Corporation.

Suitiable alkoxylated fatty alcohols for use in the present invention include, but are not limited to, alkoxylated C20-C40 fatty alcohols sold under the PERFORMATHOX® name by New Phase Technologies such as, for example, PERFORMATHOX® 420 ETHOXYLATE (Mn=575; 20% by weight ethoxylation), PERFORMATHOX® 450 ETHOXYLATE (Mn=920; 50% by weight ethoxylation), PERFORMATHOX® 480 ETHOXYLATE (Mn=2300; 80% by weight ethoxylation), PERFORMATHOX® 490 ETHOXYLATE (Mn=4600; 90% by weight ethoxylation), PERFORMATHOX® 520 ETHOXYLATE (Mn=690; 20% by weight ethoxylation), and PERFORMATHOX® 550 ETHOXYLATE (Mn=1100; 50% by weight ethoxylation).

Preferably, the alkyl ethoxylated polymer represents from about 3% to about 30% by weight of the total weight of the composition, more preferably from about 4% to about 20% by weight of the total weight of the composition, and most preferably from about 5% to about 10% by weight of the total composition, including all ranges and subranges therebetween.

Polar Modified Polymer

According to the present invention, the compositions of the present invention comprise at least one polar modified polymer. That is, the compositions comprise (1) at least one oil-soluble polar modified polymer (including one, two or more of such polymers), (2) at least one oil-soluble high carbon polar modified polymer (including one, two or more of such polymers), or (3) at least one oil-soluble polar modified polymer and at least one oil-soluble high carbon polar modified polymer (including one, two or more of each type of polymer).

Oil-Soluble High Carbon Polar Modified Polymer

According to the present invention, compositions comprising at least one oil-soluble high carbon polar modified polymer are provided. “Polar modified polymer” as used herein refers to a hydrophobic homopolymer or copolymer which has been modified with hydrophilic unit(s). “Oil-soluble” as used herein means that the polar modified polymer is soluble in oil. “High carbon” means more than 20 carbon atoms.

Suitable monomers for the hydrophobic homopolymers and/or copolymers include, but are not limited to, cyclic, linear or branched, substituted or unsubstituted, C22-C40 compounds such as, C22-C28 compounds, C24-C26 compounds, C26-C28 compounds, and C30-C38 compounds, including all ranges and subranges therebetween. Preferably, the monomers are C24-26 compounds, C26-C28 compounds or C30-C38 compounds.

Suitable hydrophilic unit(s) include, but are not limited to, maleic anhydride, acrylates, alkyl acrylates such as, for example, methyl acrylate, ethyl acrylate, propyl acrylate, and butyl acrylate, and polyvinylpyrrolidone (PVP).

According to preferred embodiments, the oil-soluble high carbon polar modified polymer is a wax. Also preferably, the oil-soluble high carbon polar modified polymer wax has one or more of the following properties:

a weight-average molecular weight Mw of less than or equal to 30 000 g/mol, preferably of 500 to 10 000 g/mol and particularly preferably of 1000 to 5,000 g/mol, including all ranges and subranges therebetween;

a number-average molecular weight Mn of less than or equal to 15 000 g/mol, preferably of 500 to 12 000 g/mol and particularly preferably of 1000 to 5000 g/mol, including all ranges and subranges therebetween;

a molar mass distribution Mw/Mn in the range from 1.5 to 10, preferably from 1.5 to 5, particularly preferably from 1.5 to 3 and especially preferably from 2 to 2.5, including all ranges and subranges therebetween; and/or

a crystallinity of 8% to 60%, preferably 9% to 40%, and more preferably 10% to 30%, including all ranges and subranges therebetween, as determined by differential scanning calorimetry.

According to preferred embodiments relating to a copolymer wax, it is preferable to have, based on the total weight of the copolymer backbone, 0.1 to 30% by weight of structural units originating from the one monomer and 70.0 to 99.9% by weight of structural units originating from the other monomer.

Waxes of the present invention can be based upon homopolymers or copolymers made, for example, by the process described in EP 571 882, the entire contents of which is hereby incorporated by reference. Suitable preparation processes include, for example, suspension polymerization, solution polymerization and gas-phase polymerization of olefins in the presence of catalysts, with polymerization in the monomers also being possible.

Oil-soluble high carbon polar modified polymer wax can be produced in a known manner from the hompopolymers and copolymers described above by oxidation with oxygen-containing gases, for example air, or by graft reaction with polar monomers, for example maleic acid or acrylic acid or derivatives of these acids. The polar modification of polyolefin waxes by oxidation with air is described, for example, in EP 0 890 583 A1, and the modification by grafting is described, for example, in U.S. Pat. No. 5,998,547, the entire contents of both of which are hereby incorporated by reference in their entirety.

Acceptable oil-soluble high carbon polar modified polymer waxes include, but are not limited to, homopolymers and/or copolymers of C24, C25 and/or C26 groups, copolymers C26, C27 and/or C28 groups, or copolymers of C30-C38 groups, which have been modified with hydrophilic units such as, for example, maleic anhydride, acrylate, methacrylate, polyvinylpyrrolidone (PVP), etc. Preferably, the oil-soluble high carbon polar modified polymer wax has from about 5% to about 30% hydrophilic units, more preferably from about 10% to about 25% hydrophilic units by weight with respect to the weight of the wax, including all ranges and subranges therebetween. Particularly preferred hydrophilically modified waxes are C26, C27 and/or C28 homopolymers and copolymers which have been modified with maleic anhydride units.

Particularly preferred oil-soluble high carbon polar modified polymer waxes for use in the present invention are C26-C28 alpha olefin maleic acid anhydride copolymer waxes commercially available from Clariant under the trade name LICOCARE or LICOCENE. Specific examples of such waxes include products marketed by Clariant under the LicoCare name having designations such as CM 401, which is a maleic anhydride modified wax having a Mw of 2025 and a crystallinilty of 11%, C30-C38 olefin/isopropylmaleate/maleic anhydride copolymer sold by Baker Hughes under the name Performa® V 1608, and C24-C26 alpha olefin acrylate copolymer wax commercially available from Clariant under the trade name LICOCARE CA301 LP3346 based on a polar backbone with C24-26 side chains with alternating ester and carboxylic acid groups.

According to other embodiments of the present invention, the polar modified polymer is not a wax. In accordance with these embodiments of the present invention, the polar modified polymer is based upon a homopolymer and/or copolymer of hydrophobic monomer(s) and has a weight-average molecular weight Mw of less than or equal to 1,000,000 g/mol, preferably of 1000 to 250,000 g/mol and particularly preferably of 5,000 to 50,000 g/mol, including all ranges and subranges therebetween.

In accordance with these embodiments, the polar modified polymer can be of any form typically associated with polymers such as, for example, block copolymer, a grafted copolymer or an alternating copolymer. For example, the polar modified polymer can contain a hydrophobic backbone (such as polypropylene and/or polyethylene) onto which hydrophilic groups (such as maleic anhydride) have been attached by any means including, for example, grafting. The attached groups can have any orienation (for example, atactic, isotactic or syndiotactic along the backbone).

Preferably, the oil-soluble high carbon polar modified polymer(s) represent from about 0.5% to about 30% of the total weight of the composition, preferably from about 1% to about 20% of the total weight of the composition, preferably from about 1.5% to about 15%, preferably from about 2% to about 8%, including all ranges and subranges therebetween.

Oil-Soluble Polar Modified Polymer

According to the present invention, compositions comprising at least one oil-soluble polar modified polymer are provided. “Polar modified polymer” as used herein refers to a hydrophobic homopolymer or copolymer which has been modified with hydrophilic unit(s). “Oil-soluble” as used herein means that the polar modified polymer is soluble in oil.

Suitable monomers for the hydrophobic homopolymers and/or copolymers include, but are not limited to, cyclic, linear or branched, substituted or unsubstituted, C2-C20 compounds such as, for example, styrene, ethylene, propylene, isopropylene, butylene, isobutylene, pentene, isopentene, isoprene, hexene, isohexene, decene, isodecene, and octadecene, including all ranges and subranges therebetween. Preferably, the monomers are C2-C8 compounds, more preferably C2-C6 compounds, and most preferably C2-C4 compounds such as ethylene, propylene and butylene.

Suitable hydrophilic unit(s) include, but are not limited to, maleic anhydride, acrylates, alkyl acrylates such as, for example, methyl acrylate, ethyl acrylate, propyl acrylate, and butyl acrylate, and polyvinylpyrrolidone (PVP).

According to the present invention, the polar modified polymer is oil-soluble: that is, the polymer does not contain a sufficient amount of hydrophilic unit(s) to render the entire polymer water-soluble or oil-insoluble. According to preferred embodiments, the polar modified polymer contains the same amount of hydrophobic monomer as hydrophilic unit (1:1 ratio) or more hydrophobic monomer than hydrophilic unit. According to particularly preferred embodiments, the polar modified polymer contains 50% or less hydrophilic unit(s) (based on weight of the polymer), 40% or less hydrophilic unit(s), 30% or less hydrophilic unit(s), 20% or less hydrophilic unit(s), 10% or less hydrophilic unit(s), 5% or less hydrophilic unit(s), 4% or less hydrophilic unit(s), or 3% or less hydrophilic unit(s).

Preferably, the polar modified polymer has from about 0.5% to about 10% hydrophilic units, more preferably from about 1% to about 8% hydrophilic units by weight with respect to the weight of the polymer, including all ranges and subranges therebetween. Particularly preferred hydrophilically modified polymers are ethylene and/or propylene homopolymers and copolymers which have been modified with maleic anhydride units.

According to preferred embodiments of the present invention, the polar modified polymer is a wax. According to particularly preferred embodiments, the polar modified wax is made via metallocene catalysis, and includes polar groups or units as well as a hydrophobic backbone. Suitable modified waxes include those disclosed in U.S. patent application publication no. 20070031361, the entire contents of which is hereby incorporated by reference. Particularly preferred polar modified waxes are C2-C3 polar modified waxes.

In accordance with preferred embodiments of the present invention, the polar modified wax is based upon a homopolymer and/or copolymer wax of hydrophobic monomers and has a weight-average molecular weight Mw of less than or equal to 25 000 g/mol, preferably of 1000 to 22 000 g/mol and particularly preferably of 4000 to 20,000 g/mol, a number-average molecular weight Mn of less than or equal to 15 000 g/mol, preferably of 500 to 12 000 g/mol and particularly preferably of 1000 to 5000 g/mol, a molar mass distribution Mw/Mn in the range from 1.5 to 10, preferably from 1.5 to 5, particularly preferably from 1.5 to 3 and especially preferably from 2 to 2.5, which have been obtained by metallocene catalysis. Also, the polar modified wax preferably has a melting point above 75° C., more preferably above 90° C. such as, for example, a melting point between 90° C. and 160° C., preferably between 100° C. and 150° C., including all ranges and subranges therebetween.

In the case of a copolymer wax, it is preferable to have, based on the total weight of the copolymer backbone, 0.1 to 30% by weight of structural units originating from the one monomer and 70.0 to 99.9% by weight of structural units originating from the other monomer. Such homopolymer and copolymer waxes can be made, for example, by the process described in EP 571 882, the entire contents of which is hereby incorporated by reference, using the metallocene catalysts specified therein. Suitable preparation processes include, for example, suspension polymerization, solution polymerization and gas-phase polymerization of olefins in the presence of metallocene catalysts, with polymerization in the monomers also being possible.

Polar modified waxes can be produced in a known manner from the hompopolymers and copolymers described above by oxidation with oxygen-containing gases, for example air, or by graft reaction with polar monomers, for example maleic acid or acrylic acid or derivatives of these acids. The polar modification of metallocene polyolefin waxes by oxidation with air is described, for example, in EP 0 890 583 A1, and the modification by grafting is described, for example, in U.S. Pat. No. 5,998,547, the entire contents of both of which are hereby incorporated by reference in their entirety.

Acceptable polar modified waxes include, but are not limited to, homopolymers and/or copolymers of ethylene and/or propylene groups which have been modified with hydrophilic units such as, for example, maleic anhydride, acrylate, methacrylate, polyvinylpyrrolidone (PVP), etc. Preferably, the C2-C3 wax has from about 0.5% to about 10% hydrophilic units, more preferably from about 1% to about 8% hydrophilic units by weight with respect to the weight of the wax, including all ranges and subranges therebetween. Particularly preferred hydrophilically modified waxes are ethylene and/or propylene homopolymers and copolymers which have been modified with maleic anhydride units.

Particularly preferred C2-C3 polar modified waxes for use in the present invention are polypropylene and/or polyethylene-maleic anhydride modified waxes (“PEMA,” “PPMA.” “PEPPMA”) commercially available from Clariant under the trade name LICOCARE or LICOCENE, Specific examples of such waxes include products marketed by Clariant under the LicoCare name having designations such as PP207.

Other suitable polar modified polymers include, but are not limited to A-C 573 A (ETHYLENE-MALEIC ANHYDRIDE COPOLYMER; Drop Point, Mettler: 106° C.) from Honeywell, A-C 596 A (PROPYLENE-MALEIC ANHYDRIDE COPOLYMER; Drop Point, Mettler: 143° C.) from Honeywell, A-C 597 (PROPYLENE-MALEIC ANHYDRIDE COPOLYMER; Drop Point, Mettler: 141° C.) from Honeywell, ZeMac® copolymers (from VERTELLUS) which are 1:1 copolymers of ethylene and maleic anhydride, polyisobutylene-maleic anhydride sold under the trade name ISOBAM (from Kuraray), polyisoprene-graft-maleic anhydride sold by Sigma Aldrich, poly(maleic anhydride-octadecene) sold by Chevron Philips Chemcial Co., poly (ethylene-co-butyl acrylate-co-maleic anhydride) sold under the trade name of Lotader (e.g. 2210, 3210, 4210, and 3410 grades) by Arkema, copolymers in which the butyl acrylate is replaced by other alkyl acrylates (including methyl acrylate [grades 3430, 4404, and 4503] and ethyl acrylate [grades 6200, 8200, 3300, TX 8030, 7500, 5500, 4700, and 4720) also sold by Arkema under the Lotader name, and isobutylene maleic anhydride copolymer sold under the name ACO-5013 by ISP.

According to other embodiments of the present invention, the polar modified polymer is not a wax. In accordance with these embodiments of the present invention, the polar modified polymer is based upon a homopolymer and/or copolymer of hydrophobic monomer(s) and has a weight-average molecular weight Mw of less than or equal to 1,000,000 g/mol, preferably of 1000 to 250,000 g/mol and particularly preferably of 5,000 to 50,000 g/mol, including all ranges and subranges therebetween.

In accordance with these embodiments, the polar modified polymer can be of any form typically associated with polymers such as, for example, block copolymer, a grafted copolymer or an alternating copolymer. For example, the polar modified polymer can contain a hydrophobic backbone (such as polypropylene and/or polyethylene) onto which hydrophilic groups (such as maleic anhydride) have been attached by any means including, for example, grafting. The attached groups can have any orienation (for example, atactic, isotactic or syndiotactic along the backbone).

Preferably, the polar modified polymer(s) represent from about 0.5% to about 30% of the total weight of the composition, preferably from about 1% to about 20% of the total weight of the composition, preferably from about 1.5% to about 15%, preferably from about 2% to about 8%, including all ranges and subranges therebetween.

Water

The composition of the present invention also contains water. Preferably, sufficient water is present to form a water-in-oil emulsion. The water is typically employed in an amount of from about 30% to about 80% by weight, such as from about 40% to about 75% by weight, such as from about 50% to about 69% by weight, including all ranges and subranges therebetween, all weights being based on the total weight of the composition.

Reaction Product

According to preferred embodiments of the present invention, the polar modified polymer is reacted with the alkyl ethoxylated polymer wax, in the presence of oil to form a first reaction product. If the reaction is conducted at a relatively high temperature (for example, above 140° C.) and for long time (for example, greater than 5 hours), a significant amount of the hydrophilic group (for example, carboxylic acid group associated with maleic anhydride groups) of the polar modifed polymer reacts with hydroxyl group(s) of the alkyl ethoxylated wax to yield a significant amount of the reaction product. If, however, the reaction is conducted at a relatively low temperature (for example, below 100° C.) and short time (for example, less than 1 hour), only a small portion of the hydrophilic group of the polar modified polymer reacts with hydroxyl group(s)of the alkyl ethoxylated polymer wax to yield a minor amount of reaction product. Depending upon desired application, a minor amount or a significant amount of the first reaction product may be desired.

Optional Ingredients

### Non-Volatile Solvent Capable of Solubilizing the Polar Modified Polymers

The cosmetic compositions of the present invention may optionally further comprise at least one non-volatile solvent capable of solubilizing the polar modified polymers. As used herein, the term “non-volatile” means having a boiling point of greater than about 100° C. The at least one non-volatile solvent typically comprises at least one non-volatile oil.

Examples of non-volatile oils that may be used in the present invention include, but are not limited to, polar oils such as:

hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;

synthetic oils or esters of formula R5COOR6 in which R5 represents a linear or branched higher fatty acid residue containing from 1 to 40 carbon atoms, including from to 19 carbon atoms, and R6 represents a branched hydrocarbon-based chain containing from 1 to 40 carbon atoms, including from 3 to 20 carbon atoms, with R6 +R7 □ 10, such as, for example, Purcellin oil (cetostearyl octanoate), isononyl isononanoate, C12 to C15 alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;

synthetic ethers containing from 10 to 40 carbon atoms;

C8 to C26 fatty alcohols, for instance oleyl alcohol; and

mixtures thereof.

The at least one non-volatile solvent, if present, is preferably present in the composition of the invention in an amount of from about 1% to about 50% by weight, such as from about 2% to about 40% by weight, such as from about 3% to about 30% by weight, including all ranges and subranges therebetween, all weights being based on the total weight of the composition.

Volatile Solvent

If present, the at least one volatile solvent may be chosen from a volatile silicone oil or a volatile non-silicone oil.

Suitable volatile silicone oils include, but are not limited to, linear or cyclic silicone oils having a viscosity at room temperature less than or equal to 6cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane (L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Suitable volatile non-silicone oils may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C8 to C16 alkanes such as C8 to C16 isoalkanes (also known as isoparaffins), isododecane, isodecane, isohexadecane, and for example, the oils sold under the trade names of Isopar or Permethyl, the C8 to C16 branched esters such as isohexyl or isodecyl neopentanoate and their mixtures. Preferably, the volatile non-silicone oils have a flash point of at least 40° C.

Non-limiting examples of volatile non-silicone oils are listed in Table 2 below.

TABLE 2

| | Flash Point |
|---|---|
| Compound | (° C.) |
| Isododecane | 43 |
| Ethanol, denatured | 13 |
| Propylene glycol n-butyl ether | 60 |
| Ethyl 3-ethoxypropionate | 58 |
| Propylene glycol methylether acetate | 46 |
| Isopar L (isoparaffin C11-C13) | 62 |
| Isopar H (isoparaffin C11-C12) | 56 |

In general, if present, the at least one volatile solvent is preferably present in the composition in an amount of from about 1 to about 20% by weight, such as from about 2 to about 15% by weight, and from about 3 to about 10% by weight, including all ranges and subranges therebetween, all weights being based on the total weight of the composition.

The composition of the present invention may also include any one, or more, optional ingredients. Examples thereof include, but are not limited to, colorants such as pigments and dyestuffs, co-solvents, plasticizers, preservatives, fillers, active ingredients, additional waxes and sunscreens.

According to particularly preferred embodiments, the composition will contain less than about 40% by weight of solids, such as less than about 38% by weight, and less than about 35% by weight, all weights based on the total weight of the composition.

It has surprisingly been discovered that the composition of the present invention forms a stable, emulsion capable of providing continuous clump free deposit. Without intending to be bound by theory, it is believed that the polymer modified polymers and alkyl ethoxylated polymer wax provide a degree of crystallinity which creates volumizing effects once deposited onto eye lashes. The hydrophilic portion of the alkyl ethoxylated polymer wax further provides smudge proof properties and ease of removal from the eye lashes. Moreover, the composition of the present invention possesses a unique texture and feel, is long wearing and easily washable without the need for having to employ conventional latex or other film forming polymers.

The rheological properties of the compositions in accordance with the present invention are determined by using a controlled stress rheometer, commercially available from TA Instruments under the name AR-G2. The samples are measured using a parallel plate having a stainless steel, cross hatched, 40 mm diameter plate. The gap is set at 1,000 microns. The desired temperature is precisely controlled by a Peltier system.

The mascara sample is transferred to the rheometer, and held at 25° C. for reaching temperature equilibrium. For a dynamic oscillation measurement, the sample is pre-sheared at a shear rate of 100 (1/second) for seconds, and followed by one minute at rest to reach equilibrium condition. The linear viscoelastic regime is determined in the oscillation strain sweep mode from 10-3 to 2×103% of strain, at a constant frequency □ of 1 rad/s. The region at which the elastic modulus or storage modulus G′ is independent of % strain with an increasing oscillation strain is defined as a linear viscoelastic regime. The critical strain value is determined from the region at which the elastic modulus or storage modulus G′ begins to be dependent of oscillation strain. After reaching this critical strain, the internal material structure is disrupted and the mascara composition is no longer under linear viscoelastic region.

Storage modulus G′ at frequency □ of 1 rad/s is used to represent the hardness of the composition. It is expected in conventional theory that when two dissimilar polar modified waxes are blended, an additive effect would be realized with respect to the storage modulus (G′) of the blend. Here, however, a surprising and unexpected synergy in storage modulus is achieved whereby the storage modulus (G′) for the blend is about four to about fifteen times higher than the G′ of each individual polar modified wax. This phenomenon can be explained by the synergistic effect of the interpolymer complex formed by the blending of the two polar modified waxes.

The composition of the present invention is characterized by a storage modulus G′ ranging from about 100 Pa to about 10,000 Pa, preferably ranging from about 500 to about 5,000 Pa, preferably ranging from about 750 to about 1500 Pa.

The shear viscosity η(\dot{γ}) of the mascara composition is measured in the flow mode. Before measuring the shear viscosity, the sample is pre-sheared at a shear rate \dot{γ} of 100 (1/second) for 30 seconds, and allowed to rest for one minute to reach equilibrium condition. Then, viscosity of the sample is measured in the continuous ramp mode from 10-3 (1/second) to 102 (1/second) for 10 minutes. A high value of the shear viscosity η(\dot{γ}) at low shear rate indicates high stability of structure at rest.

The present invention is further described in terms of the following non-limiting examples. Unless otherwise indicated, all parts and percentages are on a weight-by-weight percentage basis.

EXAMPLE 1-4

### Volumizing Mascara

| | | Comparative | Comparative | Comparative | Inventive |
|---|---|---|---|---|---|
| | | Example 1 | Example 2 | Example 3 | Example 4 |
| Phase | INCI | Wt/wt % | Wt/wt % | Wt/wt % | Wt/wt % |
| A | C20-C40 Pareth-10 | 10.00 | 10.00 | 10.00 | 10.00 |
| A | C26-C28 ALPHA OLEFIN | 10.00 | 0.00 | 0.00 | 3.33 |
| | MALEIC ACID ANHYDRIDE | | | | |
| | COPOLYMER wax | | | | |
| A | Propylene ethylene MALEIC | 0.00 | 10.00 | 0.00 | 3.33 |
| | ANHYDRIDE COPOLYMER wax | | | | |
| A | C₂₄₋₂₆ alpha olefin | 0.00 | 0.00 | 10.00 | 3.33 |
| | acrylate copolymer wax | | | | |
| A | Iron Oxides | 5.00 | 5.00 | 5.00 | 5.00 |
| B | DI Water | Q.S. | Q.S. | Q.S. | Q.S. |
| B | Pentylene Glycol | 2.00 | 2.00 | 2.00 | 2.00 |
| B | PVP | 7.50 | 7.50 | 7.50 | 7.50 |
| B | NaOH | 0.50 | 0.50 | 0.50 | 0.50 |
| C | SimetFhicone | 0.10 | 0.10 | 0.10 | 0.10 |
| D | Preservatives | 1.55 | 1.55 | 1.55 | 1.55 |
| | Total | 100.00 | 100.00 | 100.00 | 100.00 |
| | % Solid | 34.05 | 34.05 | 34.05 | 34.05 |

Procedure

In main tank A, the following were added: C20-C40 Paret-10, C26-28 α-olefin-maleic acid Anhydride copolymer wax, Propylene-ethylene-Maleic anhydride Copolymer wax and C24-26 α-olefin acrylate copolymer wax. The contents were the heated to 90° C. until all solids had melted.

When all solids had melted, contents were homogenized while Iron Oxides were being added to the batch. Batch was homogenized for at least 1 hr.

In side tank B with water bath, DI water, PVP, NaOH, Pentylene Glycol were added and mixed until homogeneous. The contents were heated to 85° C. with moderate agitation.

When both tanks were at temperature, side tank B was slowly added to main tank A while homogenizing.

After 5 minutes of homogenizing, Simethicone was added to the batch. Batch was homogenized for an addition of 30 minutes at 90° C.

Batch was cooled to 25° C.

At 35° C., preservatives were added and mixed until uniform.

Batch was poured to container at 25° C.

Rheological Values for Examples 1-4

| | | Viscosity at |
|---|---|---|
| | G′ at 0.1% | 0.1 1/s |
| Examples | strain (Pa) | (Pa · s) |
| 1 | 906.5 | 644.6 |
| 2 | 731.5 | 1225 |
| 3 | 238.7 | 29.34 |
| 4 | 3604 | 2619 |

### EXAMPLE 5

### Volumizing Mascara

| | | Example 5 |
|---|---|---|
| Phase | INCI | Wt/wt % |
| A | C20-C40 Pareth-10 | 10.00 |
| A | Linear Propylene-Ethylene- MALEIC | 16.00 |
| | ANHYDRIDE COPOLYMER | |
| A | Iron Oxides | 5.00 |
| B | DI Water | Q.S. |
| B | PVP | 5 |
| B | NaOH | 0.50 |
| B | Pentylene Glycol | 2.00 |
| C | Simethicone | 0.10 |
| D | Preservatives | 1.55 |
| | Total | 100.00% |
| | % Solids | 36.00% |

Procedure

In main tank A, the following were added: C20-C40 Paret-10, and Linear Propylene-ethylene-maleic Anhydride copolymer wax. The contents were the heated to 90° C. until all solids had melted.

When all solids had melted, contents were homogenized while Iron Oxides were being added to the batch. Batch was homogenized for at least 1 hr.

In side tank B with water bath, DI water, PVP, NaOH, Pentylene Glycol were added and mixed until homogeneous. The contents were heated to 85° C. with moderate agitation.

When both tanks were at 85° C., side tank B was slowly added to main tank A with a water bath while homogenizing at 800 rpm.

After 5 minutes of homogenizing, Simethicone was added to the batch. Batch was homogenized for an addition of 30 minutes at 85° C.

Batch was cooled to 25° C. with planetarium blade.

At 35° C., preservatives were added and mixed until uniform.

Batch was poured to container at 25° C.

Rheological Values for Example 5

| | | Viscosity |
|---|---|---|
| | G′ at 0.1% | at 0.1 1/s |
| Examples | strain (Pa) | (Pa · s) |
| 5 | 5074 | 2222.5 |

### EXAMPLE 6

### Volumizing Mascara

| | | Example 6 |
|---|---|---|
| Phase | INCI | Wt/wt % |
| A | C20-C40 Pareth-10 | 10.00 |
| A | C26-28 α-olefin-maleic acid | 10.00 |
| | anhydride copolymer wax | |
| A | Iron Oxides | 5.00 |
| B | DI Water | Q.S. |
| B | PVP | 2.00 |
| B | NaOH | 0.50 |
| B | Pentylene Glycol | 2.0 |
| C | Simethicone | 0.1 |
| D | Preservatives | 1.55 |
| | Total | 100.00% |
| | % Solids | 28.55% |

Procedure

In main tank A, the following were added: C20-C40 Paret-10, and C26-28 α-olefin-maleic acid Anhydride copolymer wax. The contents were then heated to 90° C. until all solids had melted.

When all solids had melted, contents were homogenized at 800 rpm while Iron Oxides were being added to the batch. Batch was homogenized for at least 1 hr.

In side tank B with water bath, DI water, PVP, NaOH, Pentylene Glycol were added and mixed until homogeneous. The contents were heated to 85° C. with moderate agitation.

When both tanks were at 85° C., side tank B was slowly added to main tank A while homogenizing at 800rpm.

After 5 minutes of homogenizing, Simethicone was added to the batch. Batch was homogenized for an addition of 30 minutes at 90° C. at 800 rpm.

Batch was cooled to 25° C. with planetarium blade.

At 35° C., preservatives were added and mixed until uniform.

Batch was poured to container at 25° C.

Rheological Values for Example 6

| | | Viscosity |
|---|---|---|
| | G′ at 0.1% | at 0.1 1/s |
| Examples | strain (Pa) | (Pa · s) |
| 6 | 22530 | 1250 |

### EXAMPLE 7

### Volumizing Mascara (and Comparative Examples)

| | | Comparative | Comparative | Inventive |
|---|---|---|---|---|
| | | Example 1 | Example 2 | Example 7 |
| Phase | INCI | Wt/wt % | Wt/wt % | Wt/wt % |
| A | C20-C40 Pareth-10 | 10.00 | 10.00 | 10.00 |
| A | C24-C26 α-olefin acrylate | 10.00 | 0.00 | 5.00 |
| | copolymer wax | | | |
| A | Propylene-ethylene-MALEIC | 0.00 | 10.00 | 5.00 |
| | ANHYDRIDE COPOLYMER wax | | | |
| A | Iron Oxides | 5.00 | 5.00 | 5.00 |
| B | DI Water | Q.S. | Q.S. | Q.S. |
| B | Pentylene Glycol | 2.00 | 2.00 | 2.00 |
| B | PVP | 7.50 | 7.50 | 7.50 |
| B | NaOH | 0.50 | 0.50 | 0.50 |
| C | Simethicone | 0.10 | 0.10 | 0.10 |
| D | Preservatives | 1.55 | 1.55 | 1.55 |
| | Total | 100.00 | 100.00 | 100.00 |
| | % Solid | 34.05 | 34.05 | 34.05 |

Procedure

In main tank A, the following were added: C20-C40 Paret-10, C24-26 α-olefin-acrylate copolymer wax and Propylene-ethylene-maleic anhydride Copolymer wax. The contents were the heated to 90° C. until all solids had melted.

When all solids had melted, contents were homogenized while Iron Oxides were being added to the batch. Batch was homogenized for at least 1 hr.

In side tank B with water bath, DI water, PVP, NaOH, Pentylene Glycol were added and mixed until homogeneous. The contents were heated to 85° C. with moderate agitation.

When both tanks were at temperature, side tank B was slowly added to main tank A while homogenizing.

After 5 minutes of homogenizing, Simethicone was added to the batch. Batch was homogenized for an addition of 30 minutes at 90° C.

Batch was cooled to 25° C.

At 35° C., preservatives were added and mixed until uniform.

Batch was poured to container at 25° C.

Rheological Values for Example 7 and Comparative Examples 1 and 2

| | | Viscosity |
|---|---|---|
| | G′ at 0.1% | at 0.1 1/s |
| Examples | strain (Pa) | (Pa · s) |
| Comparative 1 | 906.5 | 644.6 |
| Comparative 2 | 731.5 | 1225 |
| 7 | 4254 | 4114 |

### EXAMPLE 8-COMPARATIVE EXAMPLE

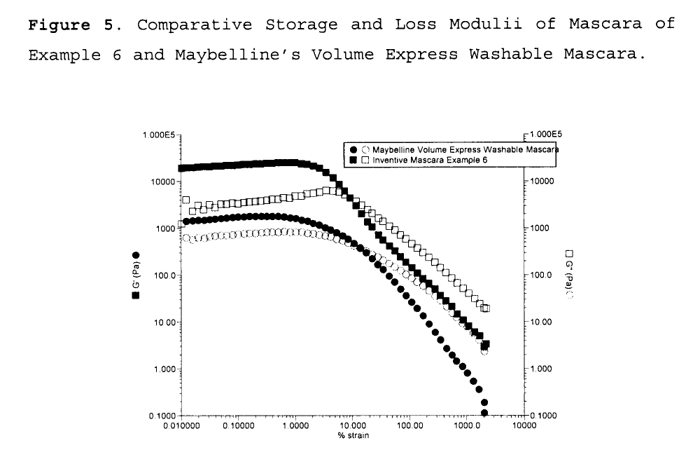

The Mascara of Example 6 was compared to commercially available Maybelline Volume Express Washable Mascara. Maybelline's Volume Express Washable Mascara has 40 to 45% solids by weight. The storage modulus of these two mascaras were measured, and the results are depicted in FIG. 5. To summarize, invention example 6 has higher modulus (G′=22530 Pa) than the Maybelline Volume Express Washable Mascara G′˜2,000 Pa). The mascara of example 6 also resulted in improving volume of the eyelashes to which it was applied.

What is claimed is:

1. A composition comprising:
(a) at least one alkyl ethoxylated polymer wax;
(b) at least one polar modified polymer; and
(c) water.

2. The composition of claim 1, further comprising at least one volatile solvent.

3. The composition of claim 1, further comprising at least one colorant.

4. The composition of claim 1, comprising less than 40% by weight solids.

5. The composition of claim 1, wherein the alkyl ethoxylated polymer wax is a Pareth-10 alcohol.

6. The composition of claim 1, wherein the composition comprises one oil-soluble polar modified polymer.

7. The composition of claim 1, wherein the composition comprises one oil soluble high carbon polymer modified polymer.

8. The composition of claim 1, wherein the composition comprises two oil soluble high carbon polymer modified polymers.

9. The composition of claim 1, wherein the composition comprises one oil-soluble polar modified polymer and one oil soluble high carbon polymer modified polymer.

10. The composition of claim 1, wherein the composition comprises one oil-soluble polar modified polymer and two oil soluble high carbon polymer modified polymers.

11. The composition of claim 1, wherein the alkyl ethoxylated polymer wax is present in an amount of from 2 to 30% by weight, based on the weight of the composition.

12. The composition of claim 1, wherein the combined amount of polar modified polymer present is in an amount of from 1 to 30% by weight, based on the weight of the composition.

13. The composition of claim 1, wherein water is present in an amount of from 30 to 80% by weight, based on the weight of the composition.

14. The composition of claim 1, further comprising at least one non-volatile solvent capable of solubilizing the polar modified polymer.

15. The composition of claim 14, wherein the non-volatile solvent is present in an amount of from 1 to 50% by weight, based on the weight of the composition.

15. The composition of claim 2, wherein the volatile solvent is present in an amount of from 1 to 20% by weight, based on the weight of the composition.

16. A method of lengthening eyelashes comprising applying onto the eyelashes the composition of claim 1.

17. A composition comprising:
(a) a reaction product of at least one alkyl ethoxylated polymer wax and at least one polar modified polymer; and
(b) water.
